(19) United States
(12) Patent Application Publication
(10) Pub. No.: US 2021/0322466 A1
(43) Pub. Date: Oct. 21, 2021

(54) METHODS AND COMPOSITION FOR IMPROVED ANTISEPSIS

(71) Applicant: Veloce BioPharma LLC, Fort Lauderdale, FL (US)

(72) Inventors: Joseph CAPRIOTTI DECIO, Arrecife (ES); Belachew TESSEMA, Framington, CT (US)

(73) Assignee: Veloce BioPharma LLC, Fort Lauderdale, FL (US)

(21) Appl. No.: 16/892,213

(22) Filed: Jun. 3, 2020

Related U.S. Application Data
(60) Provisional application No. 63/021,035, filed on May 6, 2020.
(60) Provisional application No. 63/021,039, filed on May 6, 2020.
(60) Provisional application No. 63/015,313, filed on Apr. 24, 2020.
(60) Provisional application No. 63/012,629, filed on Apr. 20, 2020.
(60) Provisional application No. 63/011,961, filed on Apr. 17, 2020.

(30) Foreign Application Priority Data
May 20, 2020 (NL) 2025640
May 20, 2020 (NL) 2025641

(51) Int. Cl. A61K 33/18; A61K 31/79; A61K 47/38; A61K 9/00; A61P 31/14

(52) U.S. Cl. CPC A61K 33/18; A61K 31/79; A61K 47/38; A61P 31/14; A61K 9/0043; A61K 9/0014; A61K 9/0053

(57) ABSTRACT

Disclosed are devices and methods for treating and disinfecting a surface that is also useful for the treatment of patients. Compositions, including antiseptic compositions, that can be used for the methods are also disclosed. In particular, invention provides an improved antiseptic with improved efficacy against COVID-19. One benefit of the disclosed methods and compositions is that it is effective even when used for very short contact times. The composition of the invention comprises an antiseptic and a cellulosic polymer.

### PRIORITY

This Application claims the benefit of priority to Netherlands Patent Application No. 2025640, filed May 20, 2020 and entitled “Methods and composition for improved antisepsis;” and Netherlands Patent Application No. 2025641, filed May 20, 2020 and entitled “Methods and compositions for improved treatment of sinus disease;” U.S. Provisional Patent Application Ser. No. 63/021,035, filed May 6, 2020 and entitled “Methods and compositions for the treatment of Covid-19;” U.S. Provisional Patent Application Ser. No. 63/021,039, filed May 6, 2020 and entitled “Methods and compositions for improved treatment of sinus disease;” U.S. Provisional Patent Application Ser. No. 63/015,313, filed Apr. 24, 2020 and entitled “Compositions and methods for improved treatment of sinus disease;” U.S. Provisional Patent Application Ser. No. 63/012,629, filed Apr. 20, 2020 and entitled “Compositions and methods for improved antiseptics;” and U.S. Provisional Patent Application Ser. No. 63/011,961, filed Apr. 17, 2020 and entitled “Compositions and methods for the treatment of Covid-19.” The entire contents of each of the above listed priority patent applications are incorporated herein by reference.

### BACKGROUND

Antiseptics are an important part of clinical medicine for their ability to decontaminate surfaces and fomites. These surfaces include living tissue such as skin, nails, epithelium and mucosal surfaces. Antiseptics are commonly used prior to routine phlebotomy, in preparation for minor and major invasive procedures, and as part of routine infection control hand-washing practices. While useful, the failure of antiseptics to fully disinfect often contributes to nosocomial infections and the spread of infectious agents. When antiseptic failure occurs, it contributes to the transmission of infectious viral particles, infectious aerosols, and viral diseases including COVID-19 which is caused by the SARS-CoV-2 virus. Failed or incomplete antisepsis of human tissues such as the nasal passages can lead to the spread of COVID-19 and can worsen outbreaks. For the same reason, failed or incomplete antisepsis of surfaces such as human skin, medical devices, tabletops, everyday objects, door handles, and any other objects touched by humans contributes to the spread of COVID-19 which is currently uncontained.

There is a long-felt clinical need for improved skin antiseptics because current skin antiseptics have a significant failure rate. Failure of current antiseptics has an adverse impact on surgical wound infection rates and infection control generally. Therefore, improved antiseptics for skin and inanimate surfaces would be useful in the reduction of transmission of the SARS-CoV-2 virus.

### SUMMARY

The present invention overcomes limitations in the prior art by providing an improved antiseptic with improved efficacy against COVID-19. One benefit of the disclosed methods and compositions is that it is effective even when used for very short contact times. Since there are no previously known povidone-iodine based antiseptics with demonstrated efficacy against the SARS-CoV-2 virus—experiments were performed to determine if this is possible.

The disclosed methods and compositions overcome limitations in the prior art by providing an improved antiseptic efficacy for compositions with increased viscosity that is effective even when used for very short contact times. An additional benefit of the disclosed methods and composition is that they are substantially less irritating to human tissues compared to other antiseptics that contain alcohol. We found that, unexpectedly, the inclusion of cellulosic polymers in small concentrations, for example, hydroxyethylcellulose at 1.0% or 1.25%, with aqueous povidone-iodine solutions of between 0.5% and 2.5% provided a composition with improved activity against many microorganisms including bacteria, fungi, biofilms and viruses; including the SARS-CoV-2 virus. Additionally, incorporating small concentrations of DMSO as a co-solvent, in a range of 2% to 30%, with aqueous povidone-iodine solutions between 0.5% and 10%, results in a dramatic improvement in the antimicrobial properties of povidone-iodine based antiseptics even in the absence of alcohols. That is, the DMSO concentration may be, for example, 2%-3%, 3%-4%, 4%-5%, 5%-6%, 6%-7%, 7%-8%, 8%-9%, 9%-10%, 10%-12%, 12%-15%, 15%-20%, 20%-25%, 25%-30% or 30%-35%. We found that these improved antiseptic compositions are able to eliminate the replication of viruses located within the sub-epithelial skin space even though the treatment involves only the surface of the skin. In addition, the effective contact time for the methods and compositions was very short. Examples of the short contact time would be, for example, less than 2 minutes, less than 1 minute, less than 30 seconds, less than 20 seconds, less than 15 seconds, less than 10 seconds and less than 5 seconds. As stated, even though the short contact times are on a surface such as skin, virus particles from sub-surface skin infections with viruses can be decontaminated. These contact times are also effective for treatment of nonhuman surfaces and can completely decontaminate the surfaces of all viruses, all bacteria, all microorganisms including the SARS-CoV-2 virus.

In one embodiment, the present invention employs a combination of a penetration enhancer and an iodophor. In one embodiment, the penetration enhancer is DMSO and the iodophor is povidone-iodine. Optionally, cellulosic polymers can be added to the composition as viscosity enhancing agents. Such a composition is ideal because it allows polyantimicrobial agents to quickly and efficiently be delivered to the skin in an effective, non-toxic concentration via a safe and convenient route.

Another embodiment relates to methods and compositions that do not include penetration enhancers (e.g., DMSO) or alcohol(s). We found surprisingly that improved chemical stability as determined by the USP titration method for povidone-iodine was obtained when aqueous PVP-I solutions of between 0.55 and 2.5% were combined with cellulosic polymers, especially hydroxyethylcellulose, in the range of 1.0% to 3.0%. In additional examples, it was surprisingly found that even compositions without DMSO were able to penetrate the nasal epithelium and effectively arrest viral replication in the nasal passages when combined with 1.0% or 1.25% HEC. This is surprising because the action of PVP-I is taught to require very high concentrations of DMSO—such as above about 40% for conventional PVP-I.

In certain embodiments, the antiseptic may comprise less than about 30%, less than 25%, less than about 20%, less than about 15%, less than about 10%, less than about 5%, or about 4% DMSO. Surprisingly, even at these low concentrations of

DMSO, in compositions with PVP-I and cellulosic polymers, these antiseptic solutions are able to penetrate through the human skin and epithelial barriers to disrupt the replication of viruses within cells.

Another aspect of the present invention involves a method for cleaning a surface comprising contacting the surface with an antiseptic of the present invention. The surface may be part of a medical device, such as, for example, a catheter, a piece of surgical equipment, an endotracheal tube, a nephrostomy tube, a biliary stent, an orthopedic device, a prosthetic valve, a medical implant, a dental device, a dental implant, a cardiac assist device, a vascular graft, a tracheostomy, an external medical device, an intrathecal device, an indwelling catheter such as a central venous catheter, a peripheral intravenous catheter, an arterial catheter, a Swan-Ganz catheter, a hemodialysis catheter, a urinary catheter, a peritoneal catheter, an umbilical catheter, a percutaneous catheter, a cuffed tunneled central venous catheter or a subcutaneous central venous port. The antiseptic can be included as a separate component of these device kits. The method of using a composition described in this invention as a nasal antiseptic in conjunction with a face mask, surgical mask, cloth covering, or other masks to prevent the spread of COVID-19 is an embodiment of the current invention. In other embodiments, the surface to be treated may be a pipe or pipeline, an oil pipeline, a water pipeline, an ice machine pipe, a beverage dispensing pipe, a floor, a tabletop, a counter-top, hospital equipment, a wheelchair, an airplane, a train, an airport check-in counter, a movie theater, a sports stadium or any other public or private surface.

In another embodiment, the surface is skin, preferably human skin. The method may comprise wiping the skin with a swab, wipe, cloth or other device (also called a delivery system) comprising the antiseptic solution. Other embodiments are directed to the use on a patient. Here, the surface may be an oral cavity, a nasal cavity, a nostril, a nasal passage, a nasopharynx or an oropharynx. In use, the method may comprise contacting the oral cavity with a mouthwash, gargle, rinse, gum or toothpaste comprising the antiseptic solution.

An aspect of the present invention relates to a swab (an example of a device or an example of a delivery system) comprising an antiseptic of the present invention. The swab may comprise a natural material (e.g., cotton) or a synthetic material. A further aspect of the present invention relates to a kit comprising the swab.

One embodiment of the invention is directed to a composition (also called the antiseptic composition) comprising an antiseptic and a cellulosic polymer. In a preferred embodiment, the recited components have a synergistic antiviral or antimicrobial effect between the cellulosic polymer and the antiseptic, or between the cellulosic polymer, the antiseptic, and the DMSO (for compositions comprising DMSO which is discussed further below). Any composition of this disclosure is considered a composition of the invention which can also be called an antiseptic composition of the invention.

One optional feature of this composition is that it does not contain a non-cellulosic polymer. The composition may be an aqueous composition comprising H₂O as a solvent. Another embodiment is directed to a device comprising the composition disclosed.

In any embodiment, the antiseptic may comprise molecular iodine, iodide, iodate, iodophor, or a combination thereof. In any embodiment, the iodophor may be povidone-iodine (PVP-I). PVP-I, or a combination thereof described above, may be present at a concentration between 0.5% and 5.0%.

In any embodiment, the cellulosic polymer may be hydroxyethylcellulose. The hydroxyethylcellulose may be at a concentration of between 0.5% and 30%.

In any embodiment, the composition may also comprise dimethylsulfoxide (DMSO). The DMSO may be at a concentration of between 0.5% and 30%.

In one embodiment, the composition may comprise 1% povidone-iodine; 5% DMSO in an aqueous solution. In one aspect, the composition may comprise 2.5% povidone-iodine; 5% DMSO, 1.25% hydroxyethylcellulose and H₂O. In one aspect, the composition may comprise 1% povidone-iodine; 5% DMSO, 1.25% hydroxyethylcellulose and H₂O to 100%.

If unspecified, any composition in this disclosure may be in an aqueous solution with H₂O filling the remaining weight to 100% (e.g., 100 wt. %). If unspecified, any percentages in this disclosure may be in weight percent (wt. %) by total weight.

Another embodiment is directed to a device comprising the antiseptic composition disclosed. The device may be a swab. The term device and delivery system is used interchangeably in this disclosure.

Another embodiment is directed to a method for reducing microorganisms on a surface of a subject comprising: applying directly to the surface an antiseptic composition which is a composition of the invention. In a preferred embodiment, the method disinfects the surface.

The microorganism, in any of the embodiments, may be a bacterium, a virus, a fungus, or a combination thereof. For example, the microorganism may be the SARS-CoV-2 virus.

The surface, in any of the embodiments, may be a surface of the sinonasal cavity, nasopharynx, oropharynx or respiratory epithelium.

The methods of the invention have the beneficial effect of reducing the number of infectious virus particles on the surface. In another embodiment, the method include reducing viral shedding, reducing viral replication and/or reducing viral transmission. One preferred method targets a virus such as the SARS-CoV-2 virus on a surface; reducing viral shedding, reducing viral replication and/or reducing viral transmission of the SARS-CoV-2 virus.

In any of the embodiments, administering may comprise contacting the antiseptic composition to the surface for a period between 1 second and 120 seconds.

Another embodiment is directed to a method for treating a microorganism infection site in a patient, comprising the step of contacting the infection site with an antiseptic composition of the invention (a composition of the invention). The infection site may be a surface. For example, the infection site may be the nasal mucosa or respiratory epithelium.

Another embodiment is directed to a method for disinfecting a surface comprising: applying directly to the surface a composition of the invention.

Another embodiment is directed to a method of disinfecting skin of a subject during an invasive procedure comprising: applying directly to the skin of the subject a composition of the invention, prior to the invasive procedure, and keeping the antiseptic composition on the skin during the invasive procedure. The invasive procedure may be a surgical, catheterization, or needle puncture procedure.

One embodiment is directed to a composition for inactivating SARS-CoV-2 virus in a virucidal assay after no more than 60 seconds of contact time between the composition and the SARS-CoV-2 virus, the composition comprises 0.5 wt %-2.5 wt % povidone-iodine; 0.15 wt %-1.25 wt % hydroxyethylcellulose and water. In a preferred embodiment, the composition comprises 1.0 wt %-2.5 wt % povidone-iodine; 1.0 wt %-1.25 wt % hydroxyethylcellulose; and water. In one embodiment, the composition does not contain DMSO. In another embodiment, the composition consists of 0.5 wt %-2.5 wt % povidone-iodine; 0.15 wt %-1.25 wt % hydroxyethylcellulose and water. In a preferred embodiment, the composition consists of 1.0 wt %-2.5 wt % povidone-iodine; 1.0 wt %-1.25 wt % hydroxyethylcellulose; and water. In one embodiment, any of these compositions, including the compositions containing “consisting of” language, the composition may optionally contain a suitable amount of pharmaceutically acceptable halide-salt to make the solution iso-osmotic with nasal mucosa.

In any of the compositions, the composition has a synergistic virucidal effect between the povidone-iodine and the hydroxyethylcellulose. That is, the composition has a virucidal effect that is greater than a sum of the virucidal effect of PVP-I alone and hydroxyethylcellulose alone. Another embodiment is directed to a method for inactivating SARS-CoV-2 in a nasopharynx, a nasal cavity, an oropharynx or an oral cavity of a subject.

The method comprising the step of administering the composition to the nasopharynx, the nasal cavity, the oropharynx or the oral cavity of the subject. Administering may comprise topical application or rinsing. In a preferred embodiment, the method reduces viral shedding of SARS-CoV-2 from the nasopharynx, the nasal cavity, the oropharynx or the oral cavity.

One embodiment is directed to a composition for inactivating SARS-CoV-2 virus in a virucidal assay after no more than 60 seconds of contact time between the composition and the SARS-CoV-2 virus, the composition comprises 0.5 wt %-2.5 wt % povidone-iodine; 0.15 wt %-1.25 wt % hydroxyethylcellulose; less than 5 wt % DMSO, and water. In a preferred embodiment, the composition comprises 1.0 wt %-2.5 wt % povidone-iodine; 1.0 wt %-1.25 wt % hydroxyethylcellulose; less than 5 wt % DMSO, and water. In another embodiment, the composition consists of 0.5 wt %-2.5 wt % povidone-iodine; 0.15 wt %-1.25 wt % hydroxyethylcellulose; less than 5 wt % DMSO, and water. In a preferred embodiment, the composition consists 1.0 wt %-2.5 wt % povidone-iodine; 1.0 wt %-1.25 wt % hydroxyethylcellulose; less than 5 wt % DMSO, and water. In any of the compositions comprising “less than 5% DMSO”, an optional minimal DMSO concentration of 0.1 wt % is envisioned. In one embodiment, any of these compositions, including the compositions containing “consisting of” language, the composition may optionally contain a suitable amount of pharmaceutically acceptable halide-salt to make the solution iso-osmotic with nasal mucosa.

In any of the compositions, the composition has a synergistic virucidal effect between the povidone-iodine and the hydroxyethylcellulose. That is, the composition has a virucidal effect that is greater than a sum of the virucidal effect of PVP-I alone and hydroxyethylcellulose alone. Also, this synergistic effect is still seen in the presence of DMSO. Another embodiment is directed to a method for inactivating SARS-CoV-2 in a nasopharynx, a nasal cavity, an oropharynx or an oral cavity of a subject. The method comprising the step of administering the composition to the nasopharynx, the nasal cavity, the oropharynx or the oral cavity of the subject. Administering may comprise, topical application or rinsing. In a preferred embodiment, the method reduces viral shedding of SARS-CoV-2 from the nasopharynx, the nasal cavity, the oropharynx or the oral cavity.

Each of the features described in this disclosure, for example, for the methods and the compositions, may be combined with any other feature unless otherwise specified.

### DETAILED DESCRIPTION OF THE INVENTION

Attempts have been made to improve antiseptics by using combinations of alcohols, bleaching agents, iodine compounds and other chemical antimicrobial agents. Viral and bacterial decontamination of skin to an acceptable and reliable level, however, is difficult to achieve. A number of reasons exist for this deficiency. One reason is that bacteria can colonize sweat glands, hair follicles, and become sequestered in layers of dead keratinized skin. Another reason is that viruses can contaminate the skin from sub-epithelial loci of infection that then shed to the skin surface where they can be transmitted. A third reason is that antiseptics can also fail to disinfect and decontaminate inanimate surfaces because of their low viscosity, poor chemical efficacy and other physical and chemical properties which render them ineffective at completely decontaminating surfaces. Further, many antiseptics require prolonged contact times with living and or inanimate surfaces to be effective and such contact times are often difficult to achieve. Thus, there remains a significant need for improved antiseptics.

An improved antiseptic could lower rates of virus transmission, viral infection, viral outbreaks, wound infection, catheter infection, and blood culture contamination, as well as reduce nosocomial contamination introduced from health care worker hands. Importantly for this invention, improved antiseptics could reduce viral contamination of surfaces and could reduce transmission of viral disease outbreaks like COVID-19. Improved antiseptics would also enable the reduced transmissibility of viral infections such as COVID-19 by reducing the number of shed virus infectious particles on surfaces, human tissues such as the nose and the mouth and the hands. Improved antiseptics in the nasal passages, as provided in the disclosed embodiments, could reduce viral transmission even when using a mask by reducing the number of viral particles in the nose. Thus, an improved antiseptic may become the standard of care for routine procedures such as putting on a surgical mask, preparing a patient for rhinoscopy or preparing a patient for a dental exam or procedure. An improved antiseptic may become the standard even when wearing a mask, when attending public events, to decontaminate surfaces in public and private spaces.

The SARS-CoV-2 virus causes the disease COVID-19. It is extremely contagious and has a worldwide distribution. It is the cause of the recent worldwide pandemic. Transmission of the infection can occur when a person has an active infection or during asymptomatic periods of viral shedding. COVID-19 is transmitted primarily through direct contact with contaminated saliva, contaminated tears, aerosolized virus particles from the mouth and aerosolized virus particles from the nose or nasopharynx or other infected secretions from one person to another. Upon exposure to COVID-19 the virus will replicate at the site of infection and can establish infection in almost any human tissue, especially in the nose, nasopharynx, mouth and oropharynx.

COVID-19 infection has a broad presentation clinically. An outbreak may be completely asymptomatic, meaning a person may be actively shedding virus without any knowledge. The infection can be symptomatic with all of the typical symptoms of viral syndromes including especially shortness of breath, dyspnea, myalgia, pyrexia, soreness, fatigue, fevers and cough. Infections can have GI symptoms like diarrhea, intolerance to food, nausea and vomiting. Seropositve individuals can be symptomatic or asymptomatic. Patients can transmit the infection to other individuals when they are symptomatic, asymptomatic or before or after they are symptomatic or asymptomatic. Transmission can occur even when wearing a mask to cover the mouth and nose. It is important to note that it is impossible to predict the number, severity or duration of outbreaks of COVID-19. Each individual experience with COVID-19 can be severe, mild or absent.

Povidone-iodine (PVP-I) is a common antiseptic with limited utility due to staining, toxicity at high concentrations and difficulty to prepare in a stable form at low concentrations. It cannot be used on mucosal surfaces at concentrations higher than 2.5%.

A variety of organic solvents are known to enhance the percutaneous absorption of medicaments, including dimethylsulfoxide (DMSO). DMSO has been shown to enhance the percutaneous penetration of many drugs. DMSO has also been shown to enhance the rate of penetration of water through the skin when the epidermis was treated for 30 minutes with 60%, 80% and 90% aqueous solutions of DMSO. Many theories concerning the mechanism of action of penetrants have appeared in the literature. One attributes the penetrant effects of DMSO, dimethylformamide, and dimethylacetamide to their hygroscopic properties which increase the water content of the stratum corneum, thereby greatly increasing its permeability. Reports of the efficacy of DMSO as a skin penetration enhancement agent require the use of high concentrations of DMSO typically above 50% and long contact times of at least 10-30 minutes. DMSO is known to be most effective with the use of only small molecules. It is previously known that DMSO is only effective at high concentrations of above 40% and only for small, uncharged molecules. In this context, PVP-I would not be considered a small uncharged molecule.

We discovered in our experiments that DMSO is effective at enhancing penetration into the stratum comeum of the skin even for large polymeric molecules such as povidone-iodine and even at low concentrations as low as 2.0%.

PVP-I, by itself, is limited as a useful antiseptic for skin and human services by limited penetration to loci of infectious agents, poor adherence in an aqueous form, low viscosity of previously known formulations, high toxicity when applied to human skin, long contact times required for antisepsis, inability to form stable formulations at low concentrations and staining of tissues and skin.

The invention relates to stable topical compositions useful in the decontamination of human tissues; decontamination of human tissue surfaces such as skin and nails; decontamination of inanimate surfaces including health care surfaces and surfaces of medical devices; decontamination of surfaces such as those found in a hospital, a medical clinic, a veterinary office, a surgical theater or other surfaces encountered in medical or surgical treatment locations; decontamination of surfaces found in a home environment such as tables, furniture, and other objects that may be found in a home; decontamination of food service areas including restaurants, kitchens and other food preparation locations; decontamination of automobiles and vehicles for personal use; decontamination of commercial transportation systems including airplanes, trains, busses and the like; decontamination of objects used by humans or animals that may become contaminated including toys, recreational objects, sports equipment, animal care objects; decontamination of any surfaces found in commercial or personal agriculture, animal care and gardening; and any and all other surfaces which may become contaminated where decontamination could be a desirable goal.

The invention also relates to stable topical compositions useful in the treatment of SARS-CoV-2 infections of the skin, mucosa, epithelium, nasal passages, oropharynx, nasopharynx, upper airway, lower airway and other human surfaces. The invention also relates to compositions and methods to reduce viral shedding, viral replication and viral transmission of SARS-CoV-2, other coronaviruses and viruses other than coronaviruses. The invention also relates to the method of reducing the amount of virus particles detectable by PCR, CC, IFA and other viral detection methods from cultured cells, from the respiratory epithelium, from the upper airway, from the lower airway and other human tissues with said compositions. The invention also relates to treating viral diseases of the respiratory epithelium, upper airway, lower airway and other human tissues with said compositions.

The invention can be a drug, antiseptic, nasal spray, intranasal gel or other pharmaceutical form and can be used in addition to a mask to prevent the spread of infectious virus particles for COVID-19. The invention can be a drug, formulation, antiseptic, nasal spray, intranasal gel or other pharmaceutical forms which can be incorporated into pads, wipes, cloths, sachets, swabs, woven materials, synthetic materials, sponges, clothing fibers, masks, gloves and other objects where reduction of contamination is desired. The methods and compositions disclosed can be used in addition to a personal protective equipment including masks to prevent the spread of infectious virus particles for COVID-19, other coronaviruses, viruses other than coronaviruses, bacteria including MRSA, Staph. spp. and any other infectious agents.

One embodiment relates to a composition that incorporates a penetration enhancer, DMSO, and an iodophor or non-iodophor antiseptic. The antiseptic is preferably povidone-iodine and a cellulosic polymer. As seen in the Examples section, the invention is surprisingly useful for the treatment of COVID-19 infection of the nasal passages, upper airway, lower airway and other human tissues. The invention is also surprisingly useful or the decontamination of surfaces after very short contact times. Examples of applicable contact times are in the range of between 5 seconds-10 seconds, between 10 seconds-15 seconds, between 15 seconds-20 seconds, between 20 seconds-30 seconds, between 30 seconds-45 seconds, and between 45 seconds-60 seconds.

A specific but non-limiting example of a formulation that leads to a useful pharmaceutical preparation consists of solid PVP-I dissolved or suspended in a 5% DMSO aqueous solution.

In another embodiment, DMSO can be added to aqueous solutions of PVP-I to produce solutions with DMSO concentrations in the range of 5%, 10%, 15% or 20%, 25%, 30% or 35%. That is, the DMSO concentration may be, for example, 2%-3%, 3%-4%, 4%-5%, 5%-6%, 6%-7%, 7%-8%, 8%-9%, 9%-10%, 10%-12%, 12%-15%, 15%-20%, 20%-25%, 25%-30% or 30%-35%. In an example DMSO can be present as a co-solvent with water in the range of 2%-40%. In any aspect of this disclosure, water is not considered a penetration enhancer. One embodiment of such a formulation could include a range of excipients such as sodium chloride, sodium dihydrogen phosphate monohydrate, cellulosic polymers like hydroxyethylcellulose, disodium hydrogen phosphate anhydrous and water, as well as others known to those skilled in the art.

In an additional embodiment, 10% PVP-I (w/v, aqueous) can be added to concentrations of DMSO aqueous solutions from 1-30% to yield a resulting solution of 1% PVP-I (w/w) with DMSO.

It is known that PVP-I aqueous solutions are difficult to stabilize at low concentrations over a long period of time. At concentrations less than about 0.7% PVP-I (w/w, aqueous), PVP-I aqueous solutions rapidly decay to yield complex mixtures of iodinated and iodine-free constituents. It is surprisingly found that in the DMSO solvent system employed in this invention, PVP-I solutions as low as 0.1% can be easily prepared and maintained as stable compositions when assessed using the USP method for PVP-I assay, for long periods of time.

It is particularly useful for the case of COVID-19 infections that stable, anhydrous or aqueous compositions that contain between 0.01%-10% PVP-I can be prepared in pure USP-grade DMSO solvents and in DMSO aqueous solutions of between 1% and 20%.

The composition and methods of this disclosure is applicable for the treatment and disinfection of many microorganisms. Non-limiting examples of each types of microorganisms are listed below.

The virus is preferably one that infects humans. The human virus is preferably selected from an adenovirus, an astrovirus, a hepadnavirus, a herpesvirus, a papovavirus, a poxvirus, an arenavirus, a bunyavirus, a calcivirus, a coronavirus, a filovirus, a flavivirus, an orthomyxovirus, a paramyxovirus, a picornavirus, a reovirus, a retrovirus, a rhabdovirus, or a togavirus. In preferred embodiments, the adenovirus includes, but is not limited to, a human adenovirus. In preferred embodiments, the astrovirus includes, but is not limited to, a mamastrovirus. In preferred embodiments, the hepadnavirus includes, but is not limited to, the hepatitis B virus. In preferred embodiments, the herpesvirus includes, but is not limited to, a herpes simplex virus type I, a herpes simplex virus type 2, a human cytomegalovirus, an Epstein-Barr virus, a varicella zoster virus, a roseolovirus, and a Kaposi's sarcoma-associated herpesvirus. In preferred embodiments, the papovavirus includes, but is not limited to, human papilloma virus and a human polyoma virus. In preferred embodiments, the poxvirus includes, but is not limited to, a variola virus, a vaccinia virus, a cowpox virus, a monkeypox virus, a smallpox virus, a pseudocowpox virus, a papular stomatitis virus, a tanapox virus, a yaba monkey tumor virus, and a molluscum contagiosum virus. In preferred embodiments, the arenavirus includes, but is not limited to lymphocytic choriomeningitis virus, a lassa virus, a machupo virus, and a junin virus. In preferred embodiments, the bunyavirus includes, but is not limited to, a hanta virus, a nairovirus, an orthobunyavirus, and a phlebovirus. In preferred embodiments, the calcivirus includes, but is not limited to, a vesivirus, a norovirus, such as the Norwalk virus and a sapovirus. In preferred embodiments, the coronavirus includes, but is not limited to, a human coronavirus (e.g., SARS-CoV-2). In preferred embodiments, the filovirus includes, but is not limited to, an Ebola virus and a Marburg virus. In preferred embodiments, the flavivirus includes, but is not limited to, a yellow fever virus, a West Nile virus, a dengue fever virus, a hepatitis C virus, a tick borne encephalitis virus, a Japanese encephalitis virus, a Murray Valley encephalitis virus, a St. Louis encephalitis virus, a Russian spring-summer encephalitis virus, a Omsk hemorrhagic fever virus, a bovine viral diarrhea virus, a Kyasanus Forest disease virus, and a Powassan encephalitis virus. In preferred embodiments, the orthomyxovirus includes, but is not limited to, influenza virus type A, influenza virus type B, and influenza virus type C. In preferred embodiments, the paramyxovirus includes, but is not limited to, a parainfluenza virus, a rubula virus (mumps), a morbillivirus (measles), a pneumovirus, such as a human respiratory syncytial virus, and a subacute sclerosing panencephalitis virus. In preferred embodiments, the picornavirus includes, but is not limited to, a poliovirus, a rhinovirus, a coxsackievirus A, a coxsackievirus B, a hepatitis A virus, an echovirus, and an eneterovirus. In preferred embodiments, the reovirus includes, but is not limited to, a Colorado tick fever virus and a rotavirus. In preferred embodiments, the retrovirus includes, but is not limited to, a lentivirus, such as a human immunodeficiency virus, and a human T-lymphotrophic virus (HTLV). In preferred embodiments, the rhabdovirus includes, but is not limited to, a lyssavirus, such as the rabies virus, the vesicular stomatitis virus and the infectious hematopoietic necrosis virus. In preferred embodiments, the togavirus includes, but is not limited to, an alphavirus, such as a Ross river virus, an O'nyong'nyong virus, a Sindbis virus, a Venezuelan equine encephalitis virus, an Eastern equine encephalitis virus, and a Western equine encephalitis virus, and a rubella virus.

Non-limiting examples of bacteria that may be treated or disinfected include Escherichia sp., Staphylococcus sp., Thermus sp., Propionibacterium sp., Rhodococcus sp., Panninobacter sp., Caulobacter sp., Brevundimonas sp., Asticcacaulis sp., Sphingomonas sp., Rhizobium sp., Ensifer sp., Bradyrhizobium sp., Tepidimonas sp., Tepidicella sp., Aquabacterium sp., Pelomonas sp., Alcaligenis sp., Achromobacter sp., Ralstonia sp., Limnobacter sp., Massilia sp., Hydrogenophaga sp., Acidovorax sp., Curvibacter sp., Delftia sp., Rhodoferax sp., Alishewanella sp., Stenotrophomonas sp., Dokdonella sp., Methylosinus sp., Hyphomicrobium sp., Methylosulfomonas sp., Methylobacteria sp., Pseudomonas sp., Enterococcus sp., Myroides sp., Burkholderia sp., Alcaligenes sp. Specific examples include Escherichia coli, Staphylococcus aureus, Pseudomonas putida, Pseudomonas mendocina, Pseudomonas oleovorans, Pseudomonas fluorescens, Pseudomonas alcaligenes, Pseudomonas pseudoalcaligenes, Pseudomonas entomophila, Pseudomonas syringae, Methylobacterium extorquens, Methylobacterium radiotolerants, Methylobacterium dichloromethanicum, Methylobacterium organophilu, Hyphomicrobium zavarzini, Enterococcus faecalis, Myroides odoratus, Pseudomonas aeruginosa, Pseudomonas orizyhabitans, Burkholderia cepacia, Alcaligenes faecalis and Sphingomonas paucimobilis.

Non-limiting examples of fungus that can be treated or disinfected include Acremonium sp., Alternaria sp., Aspergillus sp., Cladosporium sp., Fusarium sp., Mucor sp., Penicillium sp., Rhizopus sp., Stachybotrys sp., Trichoderma sp., Dematiaceae sp., Phoma sp., Eurotium sp., Scopulariopsis sp., Aureobasidium sp., Monilia sp., Botrytis sp., Stemphylium sp., Chaetomium sp., Mycelia sp., Neurospora sp., Ulocladium sp., Paecilomyces sp., Wallemia sp., Curvularia sp.

Non-limiting examples of other microorganisms that can be treated or disinfected include Saccharomycotina, Taphrinomycotina, Schizosaccharomycetes, Basidiomycota, Agaricomycotina, Tremellomycetes, Pucciniomycotina, Microbotryomycetes, Candida sp. such as Candida albicans, Candida tropicalis, Candida stellatoidea, Candida glabrata, Candida krusei, Candida guilliermondii, Candida viswanathii, Candida lusitaniae and mixtures thereof, Yarrowia sp. such as Yarrowia lipolytica, Cryptococcus sp. such as Cryptococcus gattii and Cryptococcus neofarmans, Zygosaccharomyces sp., Rhodotorula sp. such as Rhodotorula mucilaginosa.

Other non-limiting examples of microorganisms suitable for the compositions and methods include those discussed in this disclosure.

In various embodiments, the compositions encompassed herein comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

As used herein the specification, “a” or “an” may mean one or more. As used herein in the claim(s), when used in conjunction with the word “comprising,” the words “a” or “an” may mean one or more than one. As used herein, “another” may mean at least a second or more. It is contemplated that any embodiment discussed in this specification can be implemented with respect to any method or composition of the invention, and vice versa. Furthermore, compositions of the invention can be used to achieve the methods of the invention.

While the foregoing written description enables one of ordinary skill in the art to reproduce and use what is considered presently to be the best mode thereof, one of ordinary skill in the art will understand and appreciate the existence of variations, combinations, derivatives, analogs and equivalents of the specific embodiments, methods and examples provided above. The invention should therefore not be limited by the embodiments described herein, examples and methods by instead by all embodiments, examples and methods within the scope and spirit of the present invention.

Throughout this application, the term “about” is used to indicate that a value includes the inherent variation of error for the device, the method being employed to determine the value, or the variation that exists among the study subjects. The terms patient and subject are used interchangeably in this disclosure and have the same meaning.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

It is also understood that where “comprising,” “having,” “including,” or “containing” is used, compositions and methods where the word is replaced with the narrower term “consisting” is also envisioned.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### INCORPORATION BY REFERENCE

All publications, patent applications, and patents mentioned herein are hereby incorporated by reference in their entirety as if each individual publication or patent was specifically and individually indicated to be incorporated by reference. In case of conflict, the present application, including any definitions herein, will control.

### EXAMPLES

### Example 1

### Virucidal Assay of PVP-I, HEC and Aqueous Solutions of HEC/PVP-I Combined

PVP-I, HEC and combined solutions of PVP-I and HEC were tested and against SARS-CoV-2, SARS-CoV, MERS-CoV and influenza virus A (H1N1) according to virucidal quantitative suspension test EN14476.

Aqueous solutions of PVP-I 2.0%, PVP-I 1.25%, HEC 1.25%, combined PVP-I 1.25% and HEC 1.25%, and combined PVP-I 2.0% with HEC 1.25% were prepared according to previously described methods and evaluated or use as nasal antiseptic substances.

All solutions were prepared and tested at room temperature for defined contact times of 5 seconds, 15 seconds, 30 seconds and 60 seconds. Reduction in viral titer represented effective antiviral activity per European standards.

### Results

HEC 1.25% solutions were unable to inactivate SARS-CoV, SARS-CoV-2, MERS-CoV and influenza virus A (H1N1) after 5 seconds, 15 seconds, 30 seconds, or 60 seconds of exposure.

PVP-I 1.25% solutions were unable to inactivate influenza virus A (H1N1) after 5 seconds, 15 seconds or 30 seconds.

PVP-I 1.25% solutions with 1.25% HEC were unable to inactivate influenza virus A (H1N1) after contact times of 5 seconds or 15 seconds or 30 seconds.

PVP-I 1.25% solutions were unable to inactivate SARS-CoV, SARS-CoV-2, MERS-CoV after 5 seconds or 15 seconds or 30 seconds.

PVP-I 1.25% solutions were unable to inactivate SARS-CoV-2 after 5 seconds or 15 seconds.

PVP-I 1.25% solutions with 1.25% HEC were able to inactivate SARS-CoV-2 after 5 seconds, 15 seconds, 30 seconds and 60 seconds.

PVP-I 1.25% solutions with 1.25% HEC were able to inactivate SARS-CoV, MERS-CoV after 30 seconds and 60 seconds.

PVP-I 2.0% solutions were able to completely inactivate SARS-CoV, SARS-CoV-2, MERS-CoV after 60 seconds of exposure.

PVP-I 2.0% solutions were unable to inactivate SARS-CoV, SARS-CoV-2, MERS-CoV after 5 seconds, 15 seconds, or 30 seconds.

PVP-I 2.0% with 1.25% HEC solutions were unable to inactivate influenza virus A (H1N1) after contact times of 5 seconds or 15 seconds or 30 seconds.

PVP-I 2.0%/HEC 1.25% combined in a single solution inactivated influenza virus A (H1N1) after contact times of 60 seconds.

PVP-I 2.0% with HEC 1.25% solutions were able to inactivate SARS-CoV, SARS-CoV-2, MERS-CoV after 30 seconds, 60 seconds.

Aqueous solutions of HEC at 1.25% have no antiviral activity against the viruses studied.

It is surprisingly shown in the data above that 1.25% HEC has a synergistic effect on antiviral activity of 1.25% PVP-I solutions against SARS-CoV-2, less so against SARS-CoV and MERS but not any other virus tested.

It is also surprisingly found that 1.25% HEC has the strongest synergestic effect when combined with PVP-I 1.25% only against SARS-CoV-2 and not against other viruses tested. It is also surprising that the contact times required are different for each solution tested and do not follow a predictable pattern.

There is a significant and surprising synergistic effect to the combined solution of HEC and PVP-I at the concentrations studied when used against SARS-CoV-2 a but no synergy is observed against the influenza virus A (H1N1).

### Example 2

### Synergistic Effect of PVP-I and HEC when used Together Against Common Bacterial, Fungal and Viral Pathogens

Summary of Experiments: Low-dose aqueous PVP-I and polymer combinations prepared to produce topical antiseptics with better efficacy against bacteria, fungi and viruses at lower concentrations for use in human tissues such as the nasal passages. Surprisingly, I found that HEC and only HEC was able to produce a synergistic antimicrobial effect with PVP-I across a small range of concentrations but no other concentrations. This effect can be seen against all viruses and bacteria challenges. This is surprising at it was NOT seen in previous experiments against all viruses and bacteria, was not seen with other cellulosic gels, was not seen with PVP-I at greater than 5% or HEC at greater than 2.5% or less than 1.25%.

These solutions were also stable by USP titration for PVP-I at room temperature and ambient light in glass bottles and HDPE plastic for up to 6 months.

### Methods:

PVP-I aqueous solutions were prepared by adding dry PVP-I into DI water on a wt/wt basis as described. Solutions were made of aqueous PVP-I at 0.25%, 0.5%, 0.75%, 1.0%, 1.25%, 1.5%, 1.75%, 2.0%, 2.5%, 2.75%, 3.0%, 3.25%, 3.5%, 4.0%, 4.25%, 4.5%, 4.75%, 5.0%, 7.5% and 10%. These aqueous PVP-I solutions are then evaluated against MRSA biofilms in a model system as described. They are also evaluated against a range of bacterial and viral challenges as detailed below. Antibacterial, antiviral and antibiofilm activity is observed.

Next aqueous solutions are prepared with hydroxyethylcellulose 2000 cp, NF in the range of 0.25%-3.0% to produce solutions with viscosity range of 100-100 000 cps. These solutions are tested against challenge organisms as described above and against a range of bacterial, fungal and viral challenge models as described below.

Next, the above PVP-I solutions are again prepared but this time are blended with hydroxyethylcellulose 2000 cp, NF in the range of 0.25%-3.0% to produce solutions with a viscosity range of 100-100,000 cps. In these solutions, the HEC is added in place of an equivalent weight percent of water. Thus, all solutions have the same % PVP-I as the above aqueous solutions without the addition of HEC. Adjustment with NaOH or another base is used as required to maintain pH between 2.5 and 7.5. Buffers are avoided. These solutions are tested against MRSA biofilms as described above and against a range of bacterial and viral challenge models using methods well known in the art. Antibiofilm, antibacterial and antiviral activity is observed in all solutions in this example. Surprisingly, solutions of 3.0% PVP-I and below which also have a viscosity of at least 200 cps have a quantifiable stronger antiviral, antibacterial, antifungal and anti-biofilm activity than either PVP-I solutions without HEC and HEC solution without PVP-I.

This synergy at certain concentrations of PVP-I and not others only when combined with HEC at certain viscosities and not others is completely unpredictable, surprising and not known in the art.

### Experimental Results for VBP-100 and VBP-105:

VBP-105 is a solution prepared as above with 1.25% HEC, 2.5% PVP-I, qs H₂O.
VBP-100 is a solution prepared as above with 1.25% HEC, 2.0% PVP-I, qs H₂O.
HEC only in Table 2 is HEC at 1.25%.
PVP-I only in Table 1 is at 2.5%.

### The Procedure and Steps are as Follows:

Inoculated 3 ml of VBP-100 or Soln A or Soln B with ˜1.0×10⁶ CFU.
Vortexed for 30 seconds.
Time clock for sampling started after vortexing.
Removed 200 μl aliquot from vehicle immediately.
Removed 200 μl aliquots from VBP-100 at 1 minute, 2 minutes, 5 minutes and 10 minutes.
Added the 200 μl aliquots to 200 μl of 0.2 N sodium thiosulfate to inactivate the PVP-I.
Colony counts on blood agar plates using Eddy Jet 2 Spiral Plater.
Incubated plates at 37° C.
The colonies were counted using Flash and Grow System and the results are shown in Tables 1, 2, 3 below.

### Experimental Results for PVP-I, HEC and VBP-105 are Listed in the Tables Below:

TABLE 1
PVP-I 2.0% Results

| | | | | 2 | 10 |
|---|---|---|---|---|---|
| | | 30 | 60 | Min- | Min- |
| | Time 0 | Sec | Sec | utes | utes |
| 1. MRSA | 1.06 × 10⁶ | 1.11 × 10⁵ | 0.0 | 0.0 | 0.0 |
| 2. MSSA | 8.28 × 10⁶ | 5.90 × 10³ | 8.20 × 10² | 0.0 | 0.0 |
| | | | (2.91) | | |
| 3. FQ-S PA | 7.02 × 10⁵ | 4.90 × 10³ | 0.0 | 0.0 | 0.0 |
| 4. FQ-R PA | 1.71 × 10⁶ | 0.0 | 0.0 | 0.0 | 0.0 |
| 5. SM | 4.10 × 10⁶ | 3.4.0 × 10³ | 0.0 | 0.0 | 0.0 |
| 6. KP | 2.66 × 10⁶ | 2.00 × 10³ | 0.0 | 0.0 | 0.0 |
| 7. CA | 5.80 × 10⁴ | 4.00 × 10³ | 0.0 | 0.0 | 0.0 |
| 8. MRSA | 9.30 × 10⁵ | 2.03 × 10³ | 2.13 × 10² | 3.05 × 10¹ | 0.0 |
| 9. MSSA | 8.10 × 10⁵ | 4.00 × 10³ | 0.0 | 0.0 | 0.0 |
| 10. MRCNS | 9.53 × 10⁵ | 0.0 | 0.0 | 0.0 | 0.0 |
| 11. MSCNS | 2.90 × 10⁵ | 0.0 | 0.0 | 0.0 | 0.0 |
| 12. EF | 1.09 × 10⁶ | 0.0 | 0.0 | 0.0 | 0.0 |
| 13. BC | 2.80 × 10⁶ | 0.0 | 0.0 | 0.0 | 0.0 |
| 14. Fusarium | 1.40 × 10⁴ | 0.0 | 0.0 | 0.0 | 0.0 |
| sp. | | | | | |

TABLE 2
HEC 1.25% Results

| Isolate | Time 0 | 5 Minutes | 10 minutes |
|---|---|---|---|
| 1. MRSA | 2.46 × 10⁶ | 1.11 × 10⁵ | 0.0 |
| 2. MSSA | 6.27 × 10⁵ | 5.90 × 10³ | 8.20 × 10² |
| 3. FQ-S PA | 4.12 × 10⁵ | 4.90 × 10³ | 0.0 |
| 4. FQ-R PA | 6.77 × 10⁶ | 3.90 × 10⁵ | 0.0 |
| 5. SM | 9.10 × 10⁶ | 3.40 × 10³ | 0.0 |
| 6. KP | 2.99 × 10⁶ | 2.00 × 10³ | 0.0 |
| 7. CA | 8.70 × 10⁴ | 4.00 × 10³ | 0.0 |
| 8. MRSA | 8.21 × 10⁵ | 2.03 × 10³ | 2.13 × 10² |
| 9. MSSA | 7.89 × 10⁶ | 6.00 × 10³ | 0.0 |
| 10. MRCNS | 6.73 × 10⁶ | 3.88 × 10⁵ | 3.00 × 10² |
| 11. MSCNS | 2.90 × 10⁶ | 0.0 | 0.0 |
| 12. EF | 2.21 × 10⁶ | 0.0 | 0.0 |
| 13. BC | 8.99 × 10⁶ | 0.00 | 0.0 |
| 14. Fusarium sp. | 7.40 × 10⁵ | 0.0 | 0.0 |

TABLE 3
VBP-105 Results Showing Synergy of HEC and PVP-I

| | | 30 | 60 | 2 | 10 |
|---|---|---|---|---|---|
| Isolate | Time 0 | Sec | Sec | Min | Min |
| 1. MRSA | 6.06 × 10⁶ | 0.00 | 0.0 | 0.0 | 0.0 |
| 2. MSSA | 4.28 × 10⁶ | 3.98 × 10⁶ | 1.31 × 10⁶ | 9.2 × 10⁴ | 0.0 |
| 3. FQ-S PA | 7.45 × 10⁵ | 0.00 | 0.0 | 0.0 | 0.0 |
| 4. FQ-R PA | 4.89 × 10⁶ | 3.63 × 10⁶ | 2.29 × 10⁵ | 1.04 × 10⁵ | 0.0 |
| 5. SM | 4.10 × 10⁶ | 0.00 | 0.0 | 0.0 | 0.0 |
| 6. KP | 5.89 × 10⁶ | 0.00 | 0.0 | 0.0 | 0.0 |
| 7. CA | 2.80 × 10⁴ | 0.00 | 0.0 | 0.0 | 0.0 |
| 8. MRSA | 9.98 × 10⁵ | 0.00 | 0.0 | 0.0 | 0.0 |
| 9. MSSA | 9.10 × 10⁵ | 8.18 × 10⁵ | 6.10 × 10⁴ | 1.18 × 10⁴ | 0.0 |
| 10. MRCNS | 9.77 × 10⁵ | 0.00 | 0.0 | 0.0 | 0.0 |
| 11. MSCNS | 3.90 × 10⁵ | 0.00 | 0.0 | 0.0 | 0.0 |
| 12. EF | 4.19 × 10⁵ | 0.0 | 0.0 | 0.0 | 0.0 |
| 13. BC | 3.80 × 10⁶ | 0.00 | 0.0 | 0.0 | 0.0 |
| 14. Fusarium sp. | 2.54 × 10⁴ | 0.0 | 0.0 | 0.0 | 0.0 |

### Example 3

### Antifungal Studies

Evaluation of VBP-105 in vitro antifungal activity against the spores of 5 fungal strains using the Minimal Inhibitory Concentration (MIC) assay:
1. Candida auris CDC 0389 (MDR strain)
2. Trichophyton mentagrophytes MYA4439 (QC strain)
3. Microsporum canis ATCC 26299 (QC strain)
4. Candida albicans ATCC 90028 (QC strain)
5. Aspergillus fumigatus MYA3626 (QC strain)
VBP-105 vs. HEC 1.25% vs. PVP-I 2.5%
Solution stocks were prepared in appropriate solvents following the CLSI guidelines. MIC—Broth microdilution assays were performed according to the procedures detailed in CLSI document M38-A2 (CLSI, 2008) and document M27-A3.

### Reference

CLSI. 2008. Reference method for broth dilution antifungal susceptibility testing of filamentous fungi: approved standard, Second Edition CLSI document M38-A2 and document M27-A3. Clinical and Laboratory Standards Institute (CLSI).
Reference Method for Broth Dilution Antifungal Susceptibility Testing of Yeasts;
Approved Standard—Third Edition. CLSI document M27-A3.

### Results

VBP-105 demonstrated excellent antifungal activity against all the test fungal strains. At dilutions as low as and 1/16 and 1/32, VBP-105 showed antifungal activity against all the test strains. Comparator HEC was ineffective all organisms tested. PVP-I at 2% limited antifungal activity against all the strains tested but lost all efficacy at ½ dilution.

### Example 4

### Studies on 3D EPiAirway Model

The following experiment shows the effect of a composition of the invention on the SARS-CoV-2 virus.
1. We establish the EpiAirway multilayer cell culture or other multicellular cell culture with an apical surface and a basal surface.
2. The cells are infected with SARS-CoV-2 virus and establish the SARS-CoV-2 virus infection in the cell culture.
3. The shedding of SARS-CoV-2 virus through the apical surface in the cell culture is determined and confirmed by virus detection (i.e., by virus titering).
4. The apical surfaces of the model are treated with the disclosed composition for 15 seconds, 30 seconds, 45 seconds, 60 seconds or 90 seconds.
5. After the treatment, any remaining composition are rinsed away from the apical surface.
6. After a period of time between 1 h and 4 h, the amount of shedding virus are once again determined using the same method as (3). That is, the presence/amount of virus shedding through the apical surface is determined.
7. The disclosed composition is able to demonstrate a reduction in virus and virus shedding. That is, there is a 3 log reduction (99.9% reduction) in virus titer from (3) to (6) above.
8. The disclosed composition is able to demonstrate a reduction in virus and virus shedding. That is, there is a 2 log reduction (99% reduction) in virus titer from (3) to (6) above.

### Example 5

### Virucidal Efficacy of Test Compounds against SARS-CoV-2 after a 1-Minute Incubation with Virus at 22° C.+/−2° C.

The following experiments were performed and showed the effect of a composition of the invention on the SARS-CoV-2 virus. Briefly, each row represents one experiment. The indicated solution on each row was incubated for one minute with SARS-CoV-2 virus. The results are indicated on the last column where the LRV (log reduction value) of the experiment are shown.

| PVP-I | HEC | DMSO | Incubation Time | LRV^{a} |
|---|---|---|---|---|
| 2.0 | 0.0 | 0 | 1-minute | 3.98 |
| 1.25 | 1.0 | 5.0 | 1-minute | 4.63 |
| 1.25 | 1.25 | 0.0 | 1-minute | 4.63 |
| 2.0 | 0.0 | 5.0 | 1-minute | 4.00 |
| 1.25 | 0.0 | 0.0 | 1-minute | 3.99 |
| 1.25 | 1.0 | 0.0 | 1-minute | 4.63 |
| 2.5 | 1.0 | 0.0 | 1-minute | 4.63 |
| 2.5 | 1.25 | 5.0 | 1-minute | 4.63 |
| 1.5 | 1.0 | 5.0 | 1-minute | 4.63 |
| 1.25 | 1.0 | 0.0 | 1-minute | 4.63 |
| 1.75 | 1.25 | 0.0 | 1-minute | 4.63 |
| 1.75 | 1.25 | 5.0 | 1-minute | 4.63 |
| 1.25 | 1.0 | 1.0 | 1-minute | 4.63 |
| 5.0 | 1.25 | 0.0 | 1-minute | 4.63 |
| 0.0 | 1.0 | 5.0 | 1-minute | 0.63 |
| 1.0 | 1.25 | 0.0 | 1-minute | 4.63 |

^{a}LRV (log reduction value) is the reduction of virus compared to the virus control

### Example 6

### Virucidal Efficacy of Test Compounds against SARS-CoV-2 after a 1-Minute Incubation with Virus at 22° C.+/−2° C.

The following experiments were performed and showed the effect of a composition of the invention on the SARS-CoV-2 virus. Briefly, each row represents one experiment. The indicated solution on each row was incubated for one minute with SARS-CoV-2 virus. The results are indicated on the last column where the LRV (log reduction value) of the experiment are shown.

| PVP-I | HEC | DMSO | Incubation Time | LRV^{a} |
|---|---|---|---|---|
| 1.25 | 0.5 | 2.5 | 1-minute | 4.63 |
| 0.625 | 0.5 | 0.0 | 1-minute | 4.63 |
| 1.25 | 0.5 | 0.0 | 1-minute | 4.63 |
| 1.25 | 0.625 | 2.5 | 1-minute | 4.63 |
| 0.75 | 0.5 | 2.5 | 1-minute | 4.63 |
| 0.625 | 0.5 | 0.0 | 1-minute | 4.63 |
| 1.0 | 0.625 | 0.0 | 1-minute | 4.63 |
| 1.25 | 0.0 | 2.5 | 1-minute | 3.76 |
| 0.5 | 0.5 | 0 | 1-minute | 4.63 |
| 1.25 | 0.625 | 0.0 | 1-minute | 4.63 |
| 0.75 | 0.0 | 2.5 | 1-minute | 3.96 |
| 0.625 | 0.5 | 0.0 | 1-minute | 4.63 |
| 1.0 | 0.625 | 0.0 | 1-minute | 4.63 |
| 1.5 | 0.625 | 2.5 | 1-minute | 4.63 |
| 0.5 | 0.5 | 0 | 1-minute | 4.63 |
| 1.25 | 0.625 | 0.0 | 1-minute | 4.63 |

We claim:

1. A composition for inactivating SARS-CoV-2 virus in a virucidal assay after no more than 60 seconds of contact time between the composition and the SARS-CoV-2 virus, the composition comprising 0.5 wt %-2.5 wt % povidone-iodine; 0.15 wt %-1.25 wt % hydroxyethylcellulose; and water.

2. The composition of claim 1 wherein the composition does not contain DMSO.

3. The composition of claim 1 which consists of 0.5 wt %-2.5 wt % povidone-iodine; 0.15 wt %-1.25 wt % hydroxyethylcellulose; and water.

4. The composition of claim 1 which comprises a suitable amount of pharmaceutically acceptable halide-salt to make the solution iso-osmotic with nasal mucosa.

5. The composition of claim 1 wherein the composition has a synergistic virucidal effect between the povidone-iodine and the hydroxyethylcellulose.

6. A method for inactivating SARS-CoV-2 in a nasopharynx, a nasal cavity, an oropharynx or an oral cavity of a subject, the method comprising the step of administering the composition of claim 1 to the nasopharynx, the nasal cavity, the oropharynx or the oral cavity of the subject.

7. The method of claim 6 wherein administering comprises topical application or rinsing.

8. The method of claim 6 wherein the method reduces viral shedding of SARS-CoV-2 from the nasopharynx, the nasal cavity, the oropharynx or the oral cavity.

9. A composition for inactivating SARS-CoV-2 virus in a virucidal assay after no more than 60 seconds of contact time between the composition and the SARS-CoV-2 virus, the composition comprising 0.5 wt %-2.5 wt % povidone-iodine; 0.15 wt %-1.25 wt % hydroxyethylcellulose; less than 5 wt % DMSO, and water.

10. The composition of claim 9 which consists of 0.5 wt %-2.5 wt % povidone-iodine; 0.15 wt %-1.25 wt % hydroxyethylcellulose; less than 5 wt % DMSO, and water.

11. The composition of claim 9 which comprises a suitable amount of pharmaceutically acceptable halide-salt to make the solution iso-osmotic with nasal mucosa.

12. The composition of claim 9 wherein the composition has a synergistic virucidal effect between the povidone-iodine and the hydroxyethylcellulose.

13. A method for inactivating SARS-CoV-2 in a nasopharynx, a nasal cavity, an oropharynx or an oral cavity of a subject, the method comprising the step of administering the composition of claim 9 to the nasopharynx, the nasal cavity, the oropharynx or the oral cavity of the subject.

14. The method of claim 13 wherein administering comprises topical application or rinsing.

15. The method of claim 13 wherein the method reduces viral shedding of SARS-CoV-2 from the nasopharynx, the nasal cavity, the oropharynx or the oral cavity.
